(19) United States
(12) Patent Application Publication
(10) Pub. No.: US 2025/0127806 A1
(43) Pub. Date: Apr. 24, 2025

(54) INJECTABLE LIQUID PHARMACEUTICAL COMPOSITION CONTAINING GEMCITABINE AND A CYTIDINE DEAMINASE INHIBITOR

(71) Applicant: ST. JOHN'S UNIVERSITY, Queens, NY (US)

(72) Inventors: Manali Pramod Patki, Queens, NY (US); Ketankumar Dilipkumar Patel, Queens, NY (US)

(21) Appl. No.: 18/689,742

(22) PCT Filed: Oct. 26, 2022
(86) PCT No.: PCT/US2022/047811

§ 371 Date: Mar. 6, 2024

Related U.S. Application Data
(60) Provisional application No. 63/274,374, filed on Nov. 1, 2021.

(51) Int. Cl. A61K 31/7068; A61K 9/00; A61P 35/00

(52) U.S. Cl. CPC A61K 31/7068; A61K 9/0024; A61P 35/00

(57) ABSTRACT

The present invention relates to the development of a subcutaneous formulation of a combination of nucleoside, Gemcitabine and Zebularine. For immediate release formulation gemcitabine and zebularine may be formulated in saline solution. For extended-release formulation microparticles of gemcitabine and zebularine may be utilized, or a solution forming in-situ depot may be prepared.

### BACKGROUND OF THE INVENTION

Pancreatic ductal adenocarcinoma (PDAC) is a typically fatal cancer, with a prognosis of only a few years. Gemcitabine (Gem) is the first-line therapy for treating PDAC, delivered by IV infusion in a clinical setting. However, Gem's anticancer activity is severely limited by its fast inactivation by cytidine deaminase (CDA) found in pancreatic cancer cells and their microenvironment. Premature degradation of Gem necessitates a very high dose of 1000 mg/m² dose, resulting in deleterious side effects including nausea, vomiting and flu-like symptoms.

### BRIEF SUMMARY OF THE INVENTION

To solve the problem of degradation and higher drug dose, the present inventors have achieved an innovative translational approach for Gem delivery, specifically, combining Gem with a CDA inhibitor minimizes the degradation of Gem and thereby substantially reduces its dosing.

Additionally, extended-release subcutaneous depot can assist in self-administration and sustained release of the drug leading to higher patient compliance and reduced dosing frequency.

Embodiments of the present invention relates to a pharmaceutical composition of gemcitabine and zebularine, desirably for subcutaneous delivery. Such delivery may provide a therapeutically effective duration of from 0.5 h to 1 weeks.

One aspect of the invention relates to formulation of simple aqueous solution or suspension of gemcitabine and zebularine for immediate to extended release of gemcitabine and zebularine from subcutaneous tissue.

Another aspect of the invention relates to in situ injectable pharmaceutical compositions. An in situ injectable pharmaceutical composition in accordance with one embodiment of the invention includes a solution of gemcitabine and zebularine, in a mixture of a biocompatible organic solvent and a biodegradable polymer, wherein the injectable pharmaceutical composition exhibits a controlled or sustained release for 10 h to 1 week in vitro.

In accordance with embodiments of the invention, an in situ injectable pharmaceutical composition is formulated for subcutaneous, or intramuscular route of administration.

Other aspects of the invention will become apparent with the following detailed description.

### BRIEF DESCRIPTION OF THE DRAWINGS

FIG. 1 illustrates the evaluation of depot forming ability using morphological analysis and fluorescence microscopy.

FIGS. 2 (a)-(c) illustrate the determination of biodegradability of optimized formulation in tumor bearing nude mice post 1 hour (FIG. 2 (a)), 4 days (FIGS. 2 (b)) and 12 days (FIG. 2 (c)) following depot administration.

FIGS. 3 (a)-(c) illustrate the results of in vivo studies of GZxSC in nude mice bearing MIAPACA-2 xenograft tumors. FIG. 3 (a) shows the antitumor efficacy of Gemcitabine, Zebularine and GZxSC on tumor bearing mice, FIG. 3(b) shows the body weights of control and formulation treated tumor-bearing mice, and FIG. 3(c) illustrates the survival study of control and formulation treated tumor-bearing mice.

### DETAILED DESCRIPTION OF THE INVENTION

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. Please note that when a numerical range is disclosed in this description, it is intended to include all numbers within the ranges, as if each of these numbers have been individually disclosed. All publications, patent applications, patents, sequences, database entries, and other references mentioned herein are incorporated by reference in their entirety. In case of conflict, the present specification, including definitions, will control.

Pancreatic ductal adenocarcinoma (PDAC) is one of the fatal cancers, with a prognosis of only a few years. Most pancreatic cancer tumors, more than 85%, are adenocarcinomas occurring from the ductal epithelium. Lesser than 20% of cases of pancreatic cancers present with localized disease that is surgically resectable. This is majorly because the of the advanced stage of disease or it has already metastasis to other vital organs. The 5-year survival rate is less than 10%. Reports suggests that the survival rate also depends on whether the cancer originates from head or body and tail of the pancreas. It has been reported that pancreatic head cancers were associated with a significantly longer mean hospital length of stay (LOS) and higher mean cost of hospitalization compared to cancer in the body and tail. According to a recent report, mortality, new cases, and DALYs caused by pancreatic cancer has more than doubled from 1990 to 2017. A forecasting study has predicted that pancreatic cancer will be the second leading cause of cancer deaths in the USA by 2030. According to American Cancer Society, around 60,430 people will be diagnosed with pancreatic cancer in 2021. Out of which around 80% of the people will die of pancreatic cancer.

Pancreatic cancer accounts for about 3% of all cancers in the US and about 7% of all cancer deaths. Novel drug molecules and combinations have successfully improved median disease-free survival by an average of 8-20 weeks. But not much improvement in overall survival rate in past decades. Nonetheless, conventional chemotherapy typically come with inadequate outcome and serious side effects in clinical practice.

Gemcitabine (Gem), a DNA synthesis inhibitor, is still one of the first-line drug for the treatment of pancreatic cancer. Although many preclinical and clinical trials investigated chemotherapy agent to be combined with gemcitabine, only combination therapy with nab-paclitaxel has been shown to be superior to gemcitabine alone in the treatment of PDAC. Therapeutic efficacy of Gem is majorly limited by its shorter half-life, poor cellular uptake (especially when hENT1 under expressed), and rapid degradation by cytidine deaminase (CDA) enzyme present in pancreatic cancer cells and tumor associated macrophages. Rapid inactivation at tumor site necessitates high dose of Gem to achieve the anticancer effect. But dose as high as 1000 mg/m² result in severe off target side-effects. Low metronomic dosing of gemcitabine showed encouraging results in various preclinical and clinical trials. Since majority of PDAC patients are older than 55 years, very frequent intravenous administration is not a viable alternative. On the other side, molecules like gemcitabine are not suitable for extravascular administration e.g., oral delivery. Thus, a strategy to combine it with an agent potentiating anticancer activity Gem and a formulation enabling self-administration and sustained release of Gem will collectively solve the biggest issue of gemcitabine-based chemotherapy including higher systemic toxicity due to higher dosing.

Formulation/drug delivery play a key role in bioavailability/biodistribution of drug molecules and thereby clinical success e.g. Nab-paclitaxel (albumin bound paclitaxel nanoparticle) is a classic example of a formulation focused new indication of paclitaxel. Paclitaxel in Taxol formulation (ethanol+cremophor EL) is not effective in treating PDAC but in nanoform it is, due to higher tumor bioavailability and maximum tolerable dose. Combination of Nab-paclitaxel with Gem showed much higher median disease-free survival compared to individual agents and comparable to FOLFIRINOX. Although long circulation PEGylated anti-tumor agents and nanoparticle formulations have been considered to promote the overall therapeutic effectiveness, this treatment is still far from optimal due to these barriers of pancreatic cancer. Thus, a strategy boosting anticancer activity of existing drug molecule and/or novel modes of delivery to improve efficacy and patient compliance while reducing systemic side effects at the same time are need of an hour.

A variety of formulations such as nanoparticles, microspheres, implants, and hydrogels have been designed for sustained drug delivery with varying degrees of benefit. Among these in-situ forming depot have been shown to be self-administrable and easier to administer compared to preformed microparticles and implants. These are free flowable liquids at room temperature and upon injection change over into a semi-solid gel in response to the physiological fluid and temperature. This serves as a matrix for a sustained release of a drug over a period of time. It will be advantageous in minimizing hospital/outpatient visits, lower toxicity due to reduced dose, increase patient compliance and reduced overall health care cost.

In situ implants are prepared by using suitable polymer and a biocompatible solvent to solubilize the polymer. Numerous natural and synthetic biodegradable polymers such as poly-lactic-co-glycolic acid (PLGA), poly-lactic acid (PLA), poly-caprolactum (PCL), chitosan, poloxamer, and Carbopol are used for preparation of these systems. Limitations of these polymers, such as burst release, can be modified by developing block copolymers. Many synthetic biodegradable block copolymers such as poly (methacrylic acid) (PMA)-poly (ethyleneglycol) (PEG), poly (ethyleneimine), poly (l-lysine), poly (N,N-dimethyl aminoethyl methacrylamide), and ([poly (ε-caprolactone)-random-poly (D, L-lactide)]-block-poly (ethylene glycol)-block-[poly (ε-caprolactone)-random-poly (D,L-lactide)]) have been used to tailor the drug release from in situ forming systems. Choice of the solvent used in preparation of injectable in situ depot system depends on solubility of the polymer used, chemical compatibility, biocompatibility, and overall stability of the delivery system. Metabolic products of polymer or solvents used in the preparation should not have any deleterious effect on the body. Amphiphilic PCL/PEG polymer can form micro/nanoparticles or thermosensitive hydrogels, depending on the molecular weight and PCL/PEG hydrophobic/hydrophilic balance. Due to its biodegradability, good biocompatibility, low toxicity, amphiphilic property, ease to produce and low opsonization probability, the PCL-PEG-PCL copolymer is a good candidate for injectable drug delivery system.

### Selection of Biocompatible Organic Solvent

In order to select a biocompatible solvent for the development of in situ depot, we carried out saturation solubility of Gemcitabine free base in 12 different organic solvent which are safe to be used for subcutaneous administration in mice. Hydrochloride salt showed very poor solubility in organic solvent. As shown in Table 1, the saturation solubility of Gemcitabine free base was found to have the highest solubility in DMA followed by NMP which was around 616 mg/ml and 470 mg/ml respectively (Table 1). However, since NMP is already used in marketed in situ depot formulations and is FDA approved, we used it as a biocompatible solvent to prepare the various polymeric solutions.

TABLE 1
Saturation solubility of Gemcitabine free base in various solvents

| | | Solubility of Gemcitabine |
|---|---|---|
| # | Solvents | free base (mg/ml) |
| 1 | Diglyme | 3.81 ± 0.63 |
| 2 | Tetraglycol/Glycofurol | 129.49 ± 1.49 |
| 3 | Solketal | 17.73 ± 0.008 |
| 4 | Glycerol formal | 104.50 ± 2.03 |
| 5 | Tetrahydrofurfuryl alcohol | 221.25 ± 4.97 |
| 6 | Isosorbide dimethyl ether | 8.39 ± 0.40 |
| 7 | Ethyl lactate | 18.35 ± 0.34 |
| 8 | N,N Dimethylacetamide | 616.79 ± 2.55 |
| 9 | N-methyl pyrrolidone | 470.70 ± 3.56 |
| 10 | Polyethylene glycol 400 | 150.60 ± 2.89 |
| 11 | Polyethylene glycol 300 | 184.92 ± 13.26 |
| 12 | Propylene glycol | 160.31 ± 8.29 |
| 13 | Water | 21.83 ± 1.44 |

### Selection of Polymer

Five different polymers like poly (D, L-lactide-co-glycolide) (PLGA), poly (lactic acid-co-glycolic acid)-block-poly (ethylene glycol)-block-poly (lactic acid-co-glycolic acid) (PLGA-PEG-PLGA), Poly (ε-caprolactone)-poly (ethylene glycol)-poly (ε-caprolactone) (PCL-PEG-PCL), Hyaluronic Acid (HA)-Polyethylene glycol (PEG), were screened based on their depot forming ability and force of injectability. Different polymer concentration used were 15, 20, 25, 30% w/v.

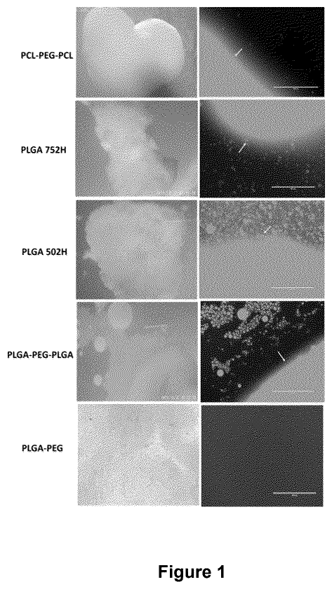

As shown in FIG. 1, PCL-PEG-PCL formed a firm depot having a uniform boundary which indicated a good forming ability of the polymer. PLGA 752H and PLGA 502H formed comparatively a good uniform depot however PLGA-PEG-PLGA formed multilocular depot and PLGA-PEG did not form a stable depot. On comparing the fluorescence images of the depot, the depot structure was ruptured, and an excess of fluorescence fragments were visible in case of PLGA 502H and PLGA-PEG-PLGA. This gives us an idea that the burst release of the drug would be more from such depot formulations. The depot prepared using PCL-PEG-PCL and PLGA 752H formed a firm uniform boundary depot and very few disrupted fragments of the dye were visible in the fluorescent images. Thus, for further characterization, we chose PCL-PEG-PCL and PLGA 752H considering their excellent and uniform depot forming ability.

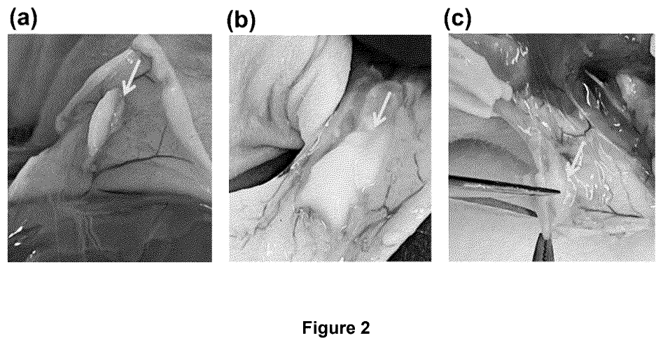

A fluorescent dye (Coumarin-6) loaded in situ depot was injected subcutaneously in pancreatic tumor (MIA-PaCa2 cells) bearing nude mice. Mice were euthanized at 1 h, 4 days and 12 days. The subcutaneous site of injection was exposed to understand and evaluate the depot formation. Later the tissue was collected and viewed under fluorescence microscope to evaluate their fluorescence intensities (FIGS. 2 (a)-(c)). The other criteria for selection of polymer were sustained release. Based on our target product profile, we wanted to sustain the drug release for up to 24 h. The depot prepared using PLGA and PCLPEGPCL released almost 75% and 42% of the hydrophilic dye respectively by end of 24 h. Thus, PCLPEGPCL sustained the dye release for longer period of time compared to PLGA and hence we selected the triblock polymer for further studies. We then used PCLPEGPCL and NMP to make drug-loaded depot formulations. We observed that the initial burst release was higher at 15% polymer concentration. This might be due to poor depot integrity at lower polymer concentration. However, at 25% polymer concentration, sustained drug release of both the drugs was observed for up to 24 h.

### Anticancer Efficacy Study of GZxSC in MIAPACA-2 Xenograft Tumors Bearing Mice

All experiments were done in accordance with the guidelines of the Institutional Animal Care and Use Committee (IACUC) at St John's university. MiaPaca-2 pancreatic cancer cells were maintained in DMEM medium supplemented with 100 U/mL penicillin, 1% streptomycin, 10% fetal bovine serum (FBS), in a humidified 5% CO₂ atmosphere at 37° C. After acclimation for 7 days, mice were subcutaneously (s.c.) injected in the right flank with Matrigel (BD Biosciences, San Jose, CA, USA) containing 1.5×10⁶ MiaPaca-2 cells. The mice were monitored closely for tumor growth by palpation and visual examination. Palpable and measurable tumors were found beginning 5-7 days after cells were injected. Tumor volume was calculated using the equation: tumor volume=(length×width×height)/2. Once the tumors reached a size of 80-120 mm³ mice were randomly divided into 4 groups to receive s.c. injection (N=6 in each group) of respective in situ depot formulation.

To evaluate antitumor efficacy, pancreatic tumor (MIA-PaCa2 cells) bearing nude mice were treated with subcutaneous in-situ forming depot formulation of Gem (10 mg/kg), Zeb (76 mg/kg) and GZxSC (Gem 10 mg/kg)+Zeb 76 mg/kg) every 3^{rd} day. Total four doses of each formulation were given. A syringe with 27G needle was used to administer 10 ul of depot formulation. Post administration, the syringe was left in the same position for 30 sec in order to obviate the sudden leakage of formulation and to facilitate the gelling of entire volumes of formulations at 37° C. Measurements of tumor size and body weight was taken every alternate day using digital calipers. Survival study was continued with rest of the animals.

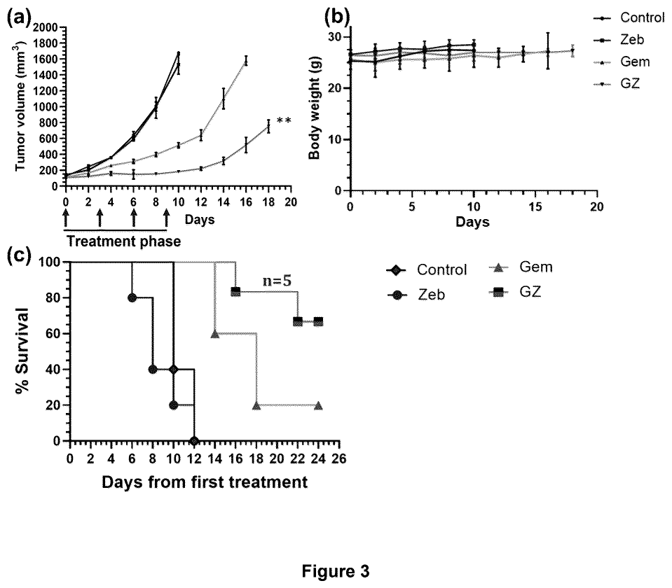

Rapid growth in tumor was observed in both control and Zeb alone group (FIG. 3(a)). Monotherapy with Zeb alone was not effective in decreasing the tumor volume and there was no significant difference between the tumor growth rate of Zeb treated group and control. Zeb alone depot did not show anti-tumor activity because dose of Zeb in our study was far below its reported anticancer activity which is more than 1000 mg/kg. Although treatment with Gem decreased the tumor volume, combination depot group-GZ showed significantly better Gem antitumor effect compared with either Gem or Zeb alone. The body weights of the tumor-bearing mice are presented in FIG. 3 (b). There were no significant differences in body weight associated with any of the dosing groups tested. Furthermore, there were no observable physiological changes, changes in mortality, or changes in the body weight after the administration of the drugs, compared to the control mice. Also, the body weights measured during the therapeutic period (Day 0-Day 9) clearly correspond to the body weight ranges of same aged control mice. All treatments were well tolerated, and no animals died during the treatment (Day 0-9). Therefore, the combination of Gem and Zeb showed a good safety profile in mice with no mortality or significant body weight loss observed. As shown in FIG. 3 (c), individual treatment only slightly increased the overall survival, but mice treated with combination had significant increase in overall survival. The median survival for control group was found to be around 10 days and for Gem group it was around 18 days. However a significant increase in the median survival of GZ group was observed (more than 24 days). All treatments were well tolerated, and no animals died during the treatment (Day 0-9). Therefore, the combination of Gem and Zeb showed a good safety profile in mice with no mortality or body weight loss.

These features provide further evidence for the improved anti-tumor efficacy. The ability to delay tumor growth in pancreatic cancer is particularly relevant as there are a significant proportion of patients with locally advanced pancreatic cancer (LAPC) or borderline pancreatic cancer (BRPC). As most pancreatic cancer patients are elderly and often have poor performance status, extended self-injectable in situ depot with low metronomic gemcitabine regimens could be advantageous compared with conventional weekly gemcitabine, if it is better tolerated and equally or more efficacious. Overall, our findings show that GZxSC treatment could be useful in the treatment of metastatic pancreatic cancer, where balancing the extension of life with the quality of life is critical.

The combination loaded in situ depot formulation was successfully prepared using biodegradable PCLPEGPCL triblock polymer and an organic solvent, NMP. It showed controlled release of both the drugs for up to 24 h and was easy to inject manually or by auto-injector using any gauge needle which would ease the process of self-administration. Our results confirm that the novel combination of Gem and Zeb shows remarkable enhancement in cytotoxicity and demonstrates a phenomenal synergistic effect. The therapeutic effect of the combination was reflected by the increased survival time of mice (>24 days) compared to control group (10 days). The result of the present study confirms that this synergistic combination delivered as a subcutaneous depot is worthy of future investigations and can be used to guide future clinical use of Gem as a potential low dose treatment of PDAC.

The present invention relates to the development of a subcutaneous formulation of a combination of nucleoside, Gemcitabine and Zebularine. For immediate release formulation gemcitabine and zebularine was formulated in saline solution. For extended-release formulation microparticles of gemcitabine and zebularine or solution forming in-situ depot was prepared. An in situ injectable pharmaceutical composition in accordance with one embodiment of the invention includes a solution of combination of gemcitabine and zebularine. Such combination may be provided in a mixture of an organic solvent and a biodegradable polymer.

The formulations of the invention contain Gemcitabine and Zebularine in any suitable concentration of 0.5-80% w/v, preferably 5-70% w/v, more preferably 10-60% w/v, most preferably 20-50% w/v.

In preferred embodiment, present composition can be injected using 18G to 31G needle, more preferably, 23G to 30G needle. The present invention will now be described by referencing the appended figures. Injectability of various embodiment prepared with a mixture of an organic solvent and a biodegradable polymer Polycaprolactone-poly (ethyleneglycol)-polycaprolactone using 25G needle.

Due to the unique combination of the organic solvent and biodegradable polymer, the formulations of the invention have the advantages of a sustained release of Gemcitabine and Zebularine which will reduce the dosing frequency. More preferably, at least two-fold reduction in dosing frequency.

In accordance with embodiments of the invention, the organic solvent is N-methyl-2-pyrrolidone, Solketal, Tetraglycol/Glycofurol, Diglyme, Glycerol formal, Tetrahydrofurfuryl alcohol, Ethyl lactate, N,N Dimethylacetamide, Isosorbide dimethyl ether, Polyethylene glycol 400, Polyethylene glycol 300, Propylene glycol, ethyl acetate, ethanol, butanol, 2-butanol, isobutanol, ispropanol, glycerin, benzyl alcohol, dimethyl sulfoxide, dimethyl glycol, an ester, an ether, an amide, a carbonate, a lactam, a sulfonyl, or any combination thereof.

In accordance with embodiments of the invention, Gemcitabine and Zebularine, together is present at a concentration of 0.25-80% w/v, preferably 0.5-70% w/v, more preferably 1-60% w/v, most preferably 2-40% w/v.

In accordance with embodiments of the invention, the biocompatible and biodegradable polymer could be PLGA, PLGA-PEG-PLGA, PCL, PCL-PEG-PCL, gelatin, alginate etc.

In accordance with embodiments of the invention, an injectable pharmaceutical composition may or may not comprise a preservative. In accordance with embodiments of the invention, the preservative that may be selected from the group consisting of benzyl alcohol, benzyl benzoate

Although the present invention is illustrated with preferred embodiment, it will be readily apparent to those with ordinary skill in the art that other embodiment may perform similar functions and/or achieve similar results. All such equivalent embodiments and examples are in the scope of the present invention and are intended to cover by following claims.

1. An injectable liquid pharmaceutical composition, comprising:
a combination of gemcitabine and a cytidine deaminase (CDA) inhibitor; and
an injectable biocompatible excipient.

2. The composition according to claim 1, wherein said cytidine deaminase (CDA) inhibitor is zebularine.

3. The composition according to claim 2, wherein said gemcitabine and zebularine are contained at a weight ratio of from 1:0.5 to 1:30.

4. The composition according to claim 3, which contains 0.25 to 50% w/w gemcitabine and zebularine.

5. The composition according to claim 4, which contains 1 to 40% w/w gemcitabine and zebularine.

6. The composition according to claim 4, wherein said biocompatible excipient comprises at least one member selected from the group consisting of water, N-methyl-2-pyrrolidone, phosphate buffer, benzyl benzoate and ethyl benzoate.

7. The composition according to claim 4, wherein said biocompatible excipient comprises a biocompatible polymer that forms a depot in situ.

8. The composition according to claim 7, wherein said depot is biodegradable.

9. The composition according to claim 8, wherein the depot provides release of gemcitabine from 1 minute to 1 week.

10. The composition according to claim 9, wherein said gemcitabine is contained in microparticle form.

11. The composition according to claim 9, wherein said biocompatible polymer comprises at least one member selected from the group consisting of poly (D,L-lactide-co-glycolide) (PLGA), poly (lactic acid-co-glycolic acid)-block-poly (ethylene glycol)-block-poly (lacticacid-co-glycolicacid) (PLGA-PEG-PLGA), and poly (ε-caprolactone)-poly (ethylene glycol)-poly (ε-caprolactone) (PCL-PEG-PCL).

12. The composition according to claim 4, which is formulated for subcutaneous administration.

13. The composition according to claim 4, which is formulated for intramuscular administration.

14. The composition according to claim 4, further comprising a preservative.

15. The composition according to claim 14, wherein said preservative comprises benzyl alcohol.

16. A pharmaceutical composition, comprising:
a combination of gemcitabine and a cytidine deaminase (CDA) inhibitor; and
a pharmaceutically acceptable excipient.

17. The composition according to claim 16, wherein said cytidine deaminase (CDA) inhibitor is zebularine.

18. The composition according to claim 17, wherein said gemcitabine and zebularine are contained at a weight ratio of from 1:0.5 to 1:30.

19. The composition according to claim 18, which contains 0.25 to 50% w/w gemcitabine and zebularine.

20. The composition according to claim 19, which contains 1 to 40% w/w gemcitabine and zebularine.

21. A method of treating a patient suffering from cancer, comprising administering gemcitabine and cytidine deaminase (CDA) inhibitor to said patient.

22. The method according to claim 21, wherein said cytidine deaminase (CDA) inhibitor is zebularine

23. The method according to claim 22, wherein said cancer is pancreatic ductal adenocarcinoma.

24. The method according to claim 22, wherein said gemcitabine and zebularine are administered at a weight ratio of from 1:0.5 to 1:30.

25. The method according to claim 24, wherein said gemcitabine and zebularine are administered simultaneously.

26. The method according to claim 24, wherein said gemcitabine and zebularine are administered at an interval.

27. The method according to claim 24, wherein said gemcitabine is administered from 1 minute to 1 week.
